(19) United States
(12) Patent Application Publication
(10) Pub. No.: US 2022/0087547 A1
(43) Pub. Date: Mar. 24, 2022

(54) ELECTRONIC DEVICE, CONTROL METHOD FOR ELECTRONIC DEVICE, AND RECORDING MEDIUM

(71) Applicant: CASIO COMPUTER CO., LTD., Tokyo (JP)

(72) Inventors: Toshihiko OTSUKA, Tokyo (JP); Takahiro TOMIDA, Tokyo (JP)

(73) Assignee: CASIO COMPUTER CO., LTD., Tokyo (JP)

(21) Appl. No.: 17/472,881

(22) Filed: Sep. 13, 2021

(30) Foreign Application Priority Data
Sep. 23, 2020 (JP) 2020-158861

(51) Int. Cl. A61B 5/026; H04N 5/235

(52) U.S. Cl. CPC A61B 5/0261; H04N 5/2353

(57) ABSTRACT

Provided are an electronic device capable of accurately measuring the effect of continuous beauty treatment based on the biological information acquired from a video, a method for controlling an electronic device, and a control program for an electronic device. The electronic device 1 includes: an imaging condition adjuster 111a that makes an imaging device capture a video of at least a part of a body to acquire pulse-wave information for adjustment indicating a pulse wave of the body based on the video, and adjusts an imaging condition based on the pulse-wave information for adjustment; and a video processing unit 111 that makes the imaging device capture a first video of at least a part of the body under the imaging condition adjusted by the imaging condition adjuster 111a to acquire video information on the body in the first video, and acquires first pulse-wave information on the body based on the video information on the body in the first video.

### CROSS-REFERENCE TO RELATED APPLICATIONS

This application is based upon and claims the benefit of priority under 35 USC 119 of Japanese Patent Application No. 2020-158861 filed on Sep. 23, 2020, the entire disclosure of which, including the description, claims, and drawings, is incorporated herein by reference in its entirety.

### BACKGROUND

### 1. Field

The present disclosure relates to electronic devices, methods for controlling an electronic device, and recording media.

### 2. Related Art

Conventionally, a technique for acquiring biological information such as blood flow of a subject from a video has been known. JP 2016-190022 describes the technique of obtaining information about subject's pulse waves based on luminance information in a video signal, and displaying the blood circulation information calculated based on the pulse wave fluctuations in the form of a heat map.

### SUMMARY

One aspect of the present invention relates to an electronic device including: a memory that stores a program; and at least one processor configured to execute the program stored in the memory. The processor is configured to adjust an imaging condition including at least one of an exposure condition of an imaging device, an amount of illumination used for the imaging device and a color of light for the illumination, based on pulse-wave information for adjustment indicating a pulse wave acquired from a video of at least a part of a subject's body captured by the imaging device, and acquire first pulse-wave information on the subject's body based on video information on the subject's body in a first video that is acquired by capturing the at least a part of the subject's body under the adjusted imaging condition.

### BRIEF DESCRIPTION OF THE DRAWINGS

FIG. 1 illustrates the configuration of a measurement system according to one embodiment of the present invention.

FIG. 2 illustrates the configuration of a measurement camera used in the measurement system according to one embodiment of the present invention.

FIG. 3 is a front view showing the external structure of the electronic device according to one embodiment of the present invention.

FIGS. 4A and 4B are side views showing the external structure of the electronic device according to one embodiment of the present invention.

FIG. 5 is a block diagram of the hardware configuration of the electronic device according to one embodiment the present invention.

FIG. 6 is a block diagram of the hardware configuration of the measurement camera used in the electronic device according to one embodiment the present invention.

FIG. 7 is a block diagram illustrating the functional configuration of the electronic device according to one embodiment of the present invention to execute measurement processing.

FIGS. 8A to 8D are graphs indicating the transition of the temporal change in the RGB luminance values before and after massage measured by the electronic device according to one embodiment of the present invention.

FIGS. 9A and 9B are graphs indicating the transition of the temporal change in the G-R values before and after massage measured by the electronic device according to one embodiment of the present invention.

FIG. 10 is a graph indicating the pulse-wave amplitude before massage measured by the electronic device according to one embodiment of the present invention.

FIGS. 11A and 11B are graphs indicating the transition of the temporal change in the amplitude PA value before and after massage measured by the electronic device according to the embodiment of the present invention.

FIG. 12 is a graph indicating the transition of the change rate of the amplitude PA value and the blood-flow baseline offset value that are measured by the electronic device according to one embodiment of the present invention.

FIGS. 13A and 13B are a graph indicating the RGB luminance value, the G luminance value, and pulse-wave amplitude and a table of the measurements measured by the electronic device according to one embodiment of the present invention.

FIGS. 14A and 14B are a graph indicating the RGB luminance value, the G luminance value, and pulse-wave amplitude and a table of the measurements measured by the electronic device according to one embodiment of the present invention.

FIGS. 15A and 15B are a graph indicating the RGB luminance value, the G luminance value, and pulse-wave amplitude and a table of the measurements measured by the electronic device according to one embodiment of the present invention.

FIGS. 16A and 16B are a graph indicating the RGB luminance value, the G luminance value, and pulse-wave amplitude and a table of the measurements measured by the electronic device according to one embodiment of the present invention.

FIGS. 17A and 17B are a graph indicating the RGB luminance value, the G luminance value, and pulse-wave amplitude and a table of the measurements measured by the electronic device according to one embodiment of the present invention.

FIGS. 18A and 18B are a graph indicating the RGB luminance value, the G luminance value, and pulse-wave amplitude and a table of the measurements measured by the electronic device according to one embodiment of the present invention.

FIGS. 19A and 19B are a graph indicating the RGB luminance value, the G luminance value, and pulse-wave amplitude and a table of the measurements measured by the electronic device according to one embodiment of the present invention.

FIG. 20 is a flowchart to explain the flow of the processing executed by the electronic device according to one embodiment of the present invention.

### DETAILED DESCRIPTION

The following describes some embodiments of the present invention, with reference to the drawings.

### Summary of One Embodiment

An electronic device 1 according to one embodiment of the present invention is a smart mirror configured as a self-supporting mirror that a user can carry. The electronic device 1 captures an image of a user who is a subject and looks in the mirror. The electronic device 1 acquires biological information based on the captured video of the user.

[System Configuration]

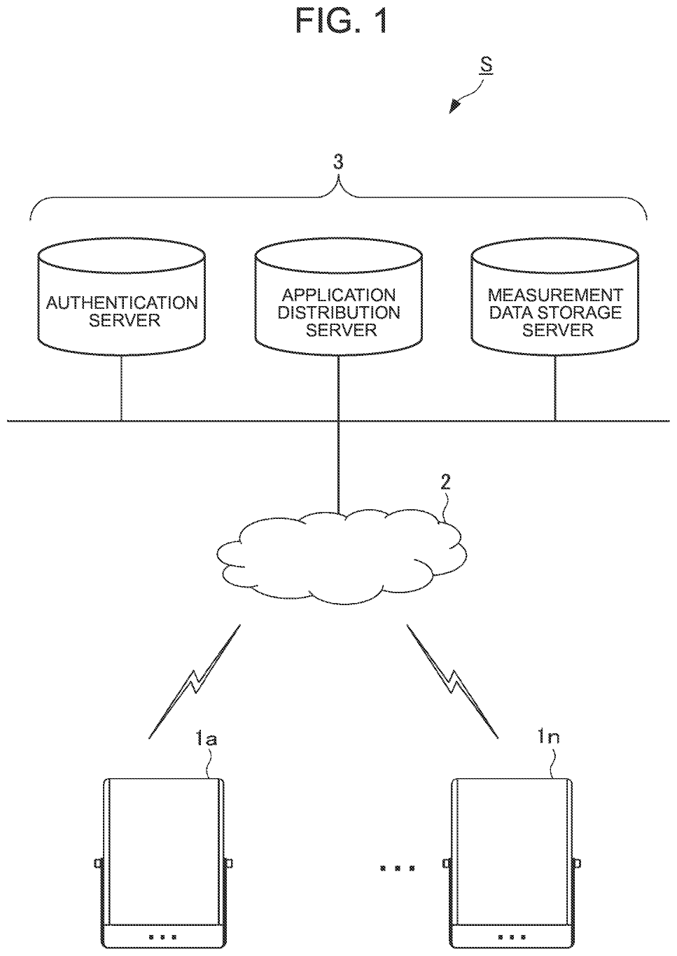

FIG. 1 is a block diagram illustrating the overall configuration of a measurement system S including the electronic device 1 according to the present embodiment. As illustrated in FIG. 1, the measurement system S includes a plurality of electronic devices 1, a network 2, and a server group 3. The number of the electronic devices 1 is not particularly limited, and n electronic devices 1 (n is any natural number) may be included in the measurement system S. In the following description, when n electronic devices 1 are described without particular distinction, they are referred to simply as an “electronic device 1” without the alphabets at the end of the reference numerals.

The electronic device 1 is a measuring device that measures a user's blood flow fluctuation from a video and displays the measurement result. The electronic device 1 is connected to the servers of the server group 3 to be communicable each other via the network 2.

In one example, the network 2 is implemented with the internet, a LAN (Local Area Network), a mobile phone network, or a network having the combination of them.

The server group 3 includes various types of servers that cooperate with the electronic device 1. In one example, the server group 3 includes an authentication server to authenticate a user of the electronic device 1. In one example, the server group 3 includes an application distribution server to distribute application software that implements the functions of the electronic device 1. In one example, the server group 3 includes a measurement data storage server that stores user profile information. The user profile information contains setting information about a user and a usage history by the user with the electronic device 1, for example.

A camera 25 dedicated to measurement (hereinafter called a measurement camera 25) is electrically connected to the electronic device 1 by wired or wireless communication.

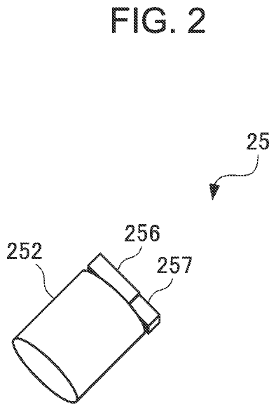

FIG. 2 illustrates the configuration of the measurement camera 25. The measurement camera 25 includes an imaging unit 256, a dedicated cover 252 that isolates the imaging unit 256 from the outside, and an illumination unit 257 that irradiates a measurement target with light inside the cover 252. The camera 25 captures an image of the subject while keeping the distal end of the cover 252 in contact with the measurement target. This keeps the brightness inside the cover constant, and thus enables the acquisition of a video while suppressing the influence from disturbance. The cover 252 includes an external-light blocking material on the inside.

The measurement system S illustrated in FIG. 1 is only one example, and the server group 3 may include servers having other functions. Each of the plurality of servers in the server group 3 may be implemented with a different server device, or the plurality of servers may be implemented with a single server device.

[External Structure]

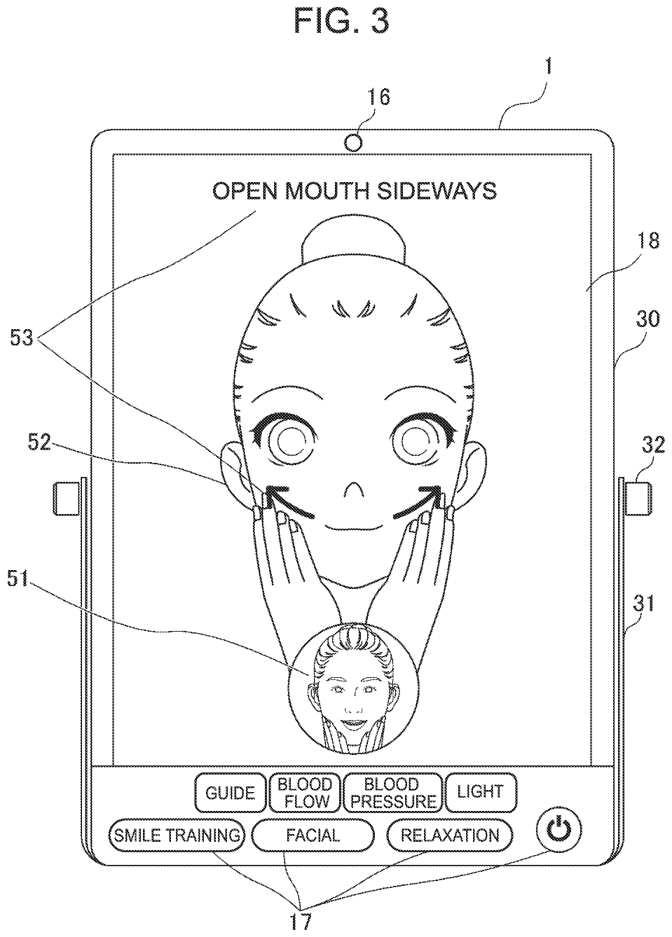

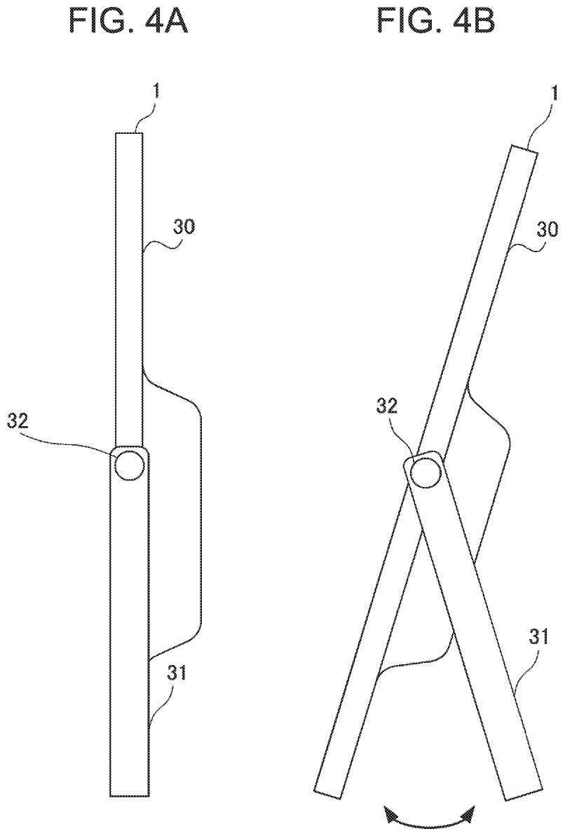

FIG. 3 is a front view showing the external structure of the electronic device 1 according to one embodiment of the present invention. FIG. 4A and FIG. 4B are side views showing the external structure of the electronic device 1. The front face of this electronic 1 has an A4 size that is defined by international organization for standardization (ISO) 216, that is an international standard.

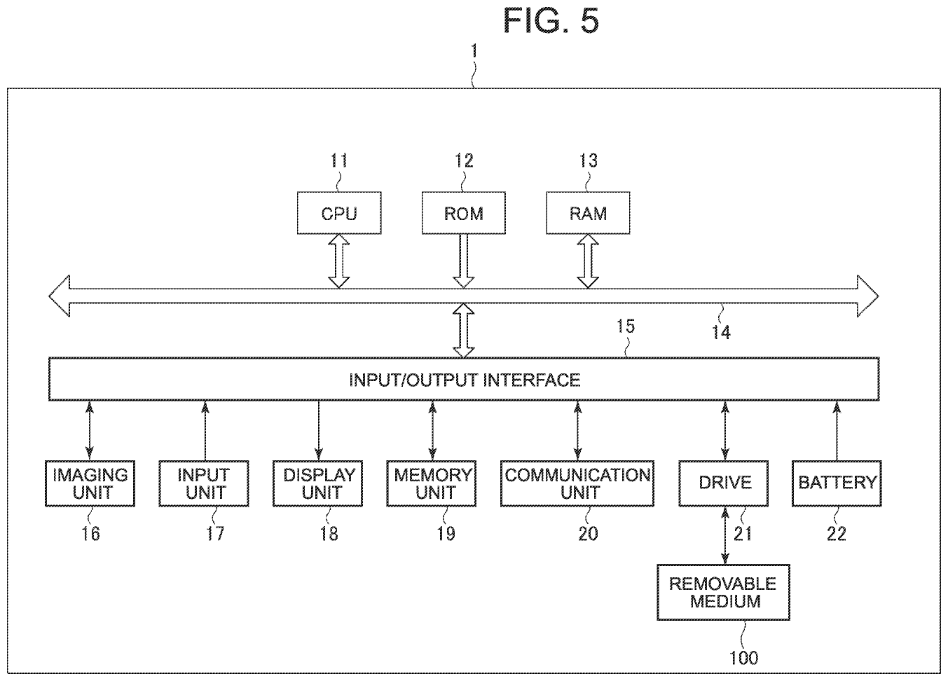

As illustrated in FIGS. 3, 4A and 4B, the electronic device 1 includes a body 30, a leg 31, and a hinge 32. The body 30 is a part including a display unit 18 and other hardware components described later with reference to FIG. 5. The leg 31 and the hinge 32 are components to make the electronic device 1 self-stand. The leg 31 is supported by the hinge 32 rotatably to the body 30.

As illustrated in FIG. 4A, before carrying the electronic device 1, the user rotates the leg 31 so that the side face of the body 30 is aligned with the side face of the leg 31 to be a compact shape for carriage. As illustrated in FIG. 4B, to place the electronic device 1 on a desk for use, the user rotates the leg 31 around the hinge 32 to make the electronic device 1 self-support. For self-supporting of the electronic device 1, the hinge 32 has a mechanism to hold the leg 31 at a predetermined angle.

The body 30 includes the display unit 18 as described above. The display unit 18 is a component that displays various kinds of information to the user. In one example, the display unit 18 displays a user image (corresponding to a user image 51 in the drawing), which is a real image of the user captured by the imaging unit 16 as a subject, an avatar image (corresponding to an avatar image 52 in the drawing), which is an alternative image of the user, and a guide image (corresponding to a guide image 53 in the drawing), which is a guide image that is auxiliary information for guidance. In this case, the display unit 18 displays the guide image synthesized with the avatar image that are displayed in a superimposed manner.

When the user sees the display unit 18, the user is allowed to understand these various pieces of information at once. As described above, the display unit 18 displays these images so that the user does not feel strangeness about the appearance, and the images have a sense of uniformity that is suitable for the user's visual recognition.

As illustrated in FIG. 3, the electronic device 1 also includes the imaging unit 16, an input unit 17, and the display unit 18 as the external structure.

The imaging unit 16 includes a camera that captures an image of the user facing to the front face of the display unit 18 during the use of the electronic device 1. The imaging unit 16 is located at a position so that the camera captures a user image 51 including the user's face facing to the front face of the display unit 18. In one example, as shown in the drawing, the imaging unit 16 is placed on the front face of the body 30 and above the display unit 18.

The electronic device 1 may capture the user image 51 with the imaging unit 16 or with the measurement camera 25 described above. Specifically, the electronic device 1 may selectively use one of the imaging unit 16 and the measurement camera 25, or may use both of them depending on the purpose.

The input unit 17 receives the input operation from the user. In one example, the input unit 17 includes a plurality of buttons. The drawing illustrates various buttons, including buttons for esthetic treatment for small-face and smile training, switching buttons for various modes such as recording of biological information, and a button for turning on/off of the electronic device 1.

That is the description on the external structure of the electronic device 1. The structure as stated above is only an example, and the external structure of the electronic device 1 is not limited to the example.

For example, the electronic device 1 may further include a light emitting unit that emits light to illuminate the user facing to the front face of the display unit 18. As the light emitting unit illuminates the user while adjusting the illumination intensity and color components, the electronic device 1 functions as an illuminating mirror. The electronic device 1 may include a plurality of these light emitting units. The light emitting unit may be placed above or below the display unit 18, or may be placed around the entire display unit 18.

In another example, the number and the location of the input unit 17 may be changed. For example, a part of the display unit 18 may include a touch panel, in which the input unit 17 and the display unit 18 are integrally configured.

[Hardware Configuration]

FIG. 5 is a block diagram illustrating the hardware configuration of the electronic device 1.

As illustrated in FIG. 5, the electronic device 1 includes a central processing unit (CPU) 11 as a processor, a read only memory (ROM) 12, a random access memory (RAM) 13, a bus 14, an input/output interface 15, the imaging unit 16, the input unit 17, the display unit 18, a memory unit 19, a communication unit 20, a drive 21 and a battery 22.

The CPU 11 executes various types of processing in accordance with a program recorded in the ROM 12 or a program loaded in the RAM 13 from the memory unit 19.

The RAM 13 stores data required to execute various types of processing by the CPU 11 as needed.

The CPU 11, the ROM 12 and the RAM 13 are mutually connected via the bus 14. The input/output interface 15 also is connected to this bus 14. To the input/output interface 15, the imaging unit 16, the input unit 17, the display unit 18, the memory unit 19, the communication unit 20, the drive 21 and the battery 22 are connected.

Although not illustrated, the imaging unit 16 includes an optical lens unit and an image sensor. The optical lens unit includes a lens to collect light, such as a focus lens or a zoom lens to capture an image of the subject. The focus lens is to form an image of the subject on a receiving surface of the image sensor. The zoom lens is to change the focal length freely in a certain range. The imaging unit 16 may further include a peripheral circuit to adjust setting parameters, such as a focal point, exposure, or white balance, as needed.

The image sensor includes a photoelectric conversion element, and an analog front end (AFE). In one example, the photoelectric conversion element may include a complementary metal oxide semiconductor (CMOS) type photoelectric conversion element. An image of the subject is incident on the photoelectric conversion element from the optical lens unit. The photoelectric conversion element then photoelectric-converts (imaging) the image of the subject to store an image signal for a certain time period, and sequentially supplies the stored image signal to the AFE as an analog signal. The AFE executes various types of signal processing of this analog image signal, such as analog/digital (A/D) conversion. The AFE creates a digital signal through the various types of signal processing, and the imaging unit 16 outputs this digital signal as an output signal. Such an output signal from the imaging unit 16 is supplied to the CPU 11, for example, as needed.

The input unit 17 includes various buttons and a microphone, for example, with which a user inputs various types of information for instruction through the operation of the buttons and the voice.

The display unit 18 includes a liquid crystal display, for example, and displays an image corresponding to the image data output from the CPU 11.

The memory unit 19 includes a semiconductor memory, such as a dynamic random access memory (DRAM), and stores various types of data.

The communication unit 20 controls communications so as to allow the CPU 11 to communicate with other devices (e.g., each server in the server group 3) via the network 2.

The drive 21 includes an interface, to which a removable medium 100 can be connected. The removable medium 100 may be connected to the drive 21 as needed, and the examples of the removable medium 100 include a magnetic disk, an optical disk, a magneto-optical disk, and a semiconductor memory. The removable media 100 stores a program for executing synthesized display processing described below and various types of data such as image data. The drive 21 may read programs and various types of data such as image data from the removable medium 100 and install them in the memory unit 19 as needed.

The battery 22 supplies electricity to various parts of the device, and is rechargeable in response to the connection to an external power source. When the electronic device 1 is not connected to an external power source, the electronic device 1 operates with the electricity from the battery 22.

The electronic device 1 may include other hardware components in addition to the above-mentioned hardware components. In one example, the electronic device 1 may further include an output unit that includes a lamp, a speaker or a vibration motor, for example, to output light, sound and a vibration signal.

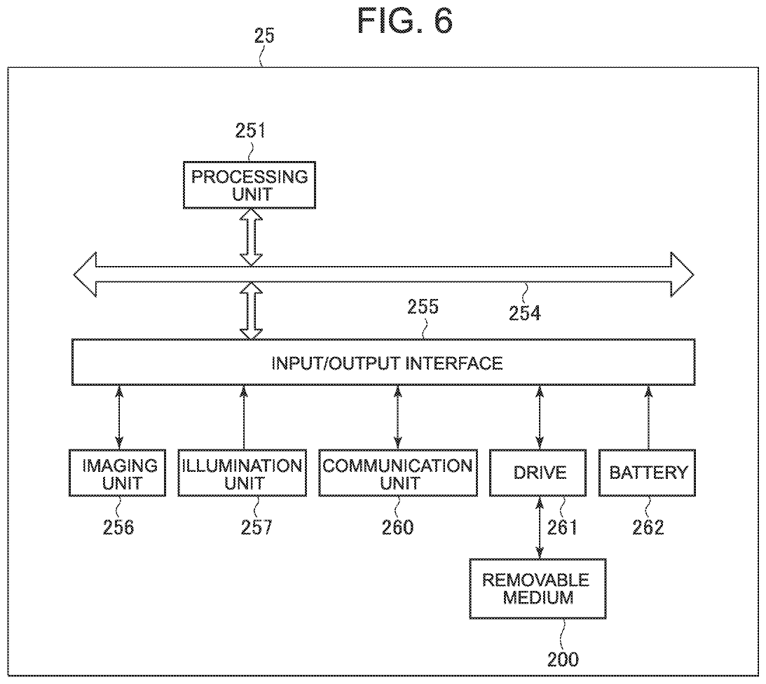

FIG. 6 is a block diagram illustrating the hardware configuration of the measurement camera 25.

As illustrated in FIG. 6, the measurement camera 25 includes a processing unit 251, an imaging unit 256, an illumination unit 257, a communication unit 260, a drive 261, and a battery 262.

The processing unit 251 controls the entire measurement camera 25. Particularly, the processing unit 251 controls the entire measurement camera 25 through a communication with the CPU 11 of the electronic device 1 via the communication unit 260 described below to acquire command signals from the electronic device 1.

This control of the entire measurement camera 25 includes the processing of videos captured by the imaging unit 256 described below.

Although not illustrated, the imaging unit 256 includes an optical lens unit and/or an image sensor. The optical lens unit includes a lens to collect light, such as a focus lens or a zoom lens, to capture an image of the subject. The focus lens is to form an image of the subject on a receiving surface of the image sensor. The zoom lens is to change the focal length freely in a certain range. The imaging unit 256 may further include a peripheral circuit to adjust setting parameters, such as a focal point, exposure, or white balance as needed. The setting parameters include control parameters of the imaging unit 256 itself that are parameters related to zoom, focus, exposure, deep-focus, and tilt, and image adjustment parameters that relate to brightness, contrast, vividness, sharpness, white balance, backlight compensation and gain.

The image sensor includes a photoelectric conversion element, and an analog front end (AFE). In one example, the photoelectric conversion element may include a complementary metal oxide semiconductor (CMOS) type photoelectric conversion element. An image of the subject is incident on the photoelectric conversion element from the optical lens unit. The photoelectric conversion element photoelectric-converts (imaging) the image of the subject to store an image signal for a certain time period, and sequentially supplies the stored image signal to the AFE as an analog signal. The AFE executes various types of signal processing of this analog image signal, such as analog/digital (A/D) conversion. The AFE creates a digital signal through the various types of signal processing, and the imaging unit 256 outputs this digital signal as an output signal. Such an output signal from the imaging unit 256 is supplied to the processing unit 251, for example, as needed.

For capturing an image by the imaging unit 256, the illumination unit 257 irradiates the field of view of the imaging unit 256 with light according to a control signal from the processing unit 251. In one example, the illumination unit 257 may be implemented with an LED and a light-control circuit. Some of the light-control circuits adjust the amount of light by the method of generating a PWM signal as the output and changing the duty ratio. If the changed duty ratio has a band frequency that is the same as or close to the frequency of the pulse wave component, this becomes noise, so that this method may adversely affect the SN ratio of the pulse wave signal. Desirably the illumination unit 257 therefore includes a light-control circuit of a constant voltage drive type.

The communication unit 260 controls communications so as to allow the processing unit 251 to communicate with the electronic device 1. The communication unit 260 may include a wireless circuit that allows the processing unit 251 to wirelessly communicate with the electronic device 1.

The drive 261 includes an interface, to which a removable medium 200 can be connected. The removable medium 200 may be connected to the drive 261 as needed, and the examples of the removable medium 200 include a semiconductor memory such as universal serial bus (USB) memory. The removable medium 200 stores various types of data such as image data and a program used for processing at the processing unit 251.

The battery 262 supplies electricity to various parts of the device, and is rechargeable in response to the connection to an external power source. When the measurement camera 25 is not connected to an external power source, the electronic device 1 operates with the electricity from the battery 262.

The measurement camera 25 may include other hardware components in addition to the above-mentioned hardware components.

[Functional Configuration]

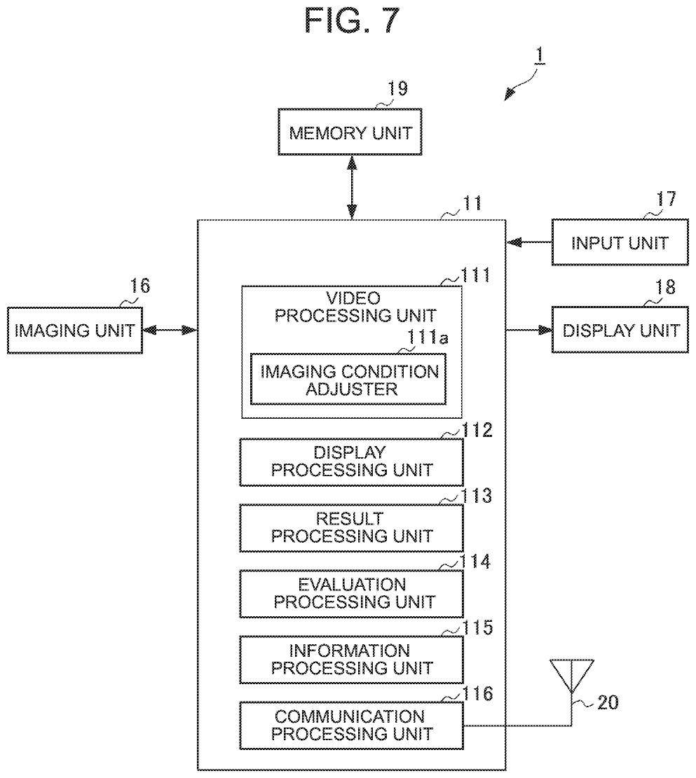

FIG. 7 is a block diagram illustrating the functional configuration of the electronic device 1 to execute measurement processing. The measurement processing is a series of processes in which the electronic device 1 displays the measurement result based on a change in the biological information values acquired from the user.

Firstly, the following describes the memory unit 19 that stores various types of information. In one example, the memory unit 19 stores various types of data related to guidance in the display process, various types of data related to an avatar that serves as a substitute for the actual image of the user, information to perform measurements, information to display measurement results, and information indicating measurement results. These various types of data may be stored only in the memory unit 19, or these data may be stored by the drive 21 in the removable medium 100 as needed. In one example, such information may be stored in the measurement data storage server in the server group 3, as needed.

Next, the following describes various functional blocks that execute the measurement processing. As illustrated in FIG. 7, the CPU 11 as a control unit functions as a video processing unit 111, a display processing unit 112, a result processing unit 113, an evaluation processing unit 114, an information processing unit 115, and a communication processing unit 116.

The video processing unit 111 analyzes a video having the user as the subject captured by the imaging unit 16 to acquire information about the user (hereinafter, referred to as “subject information”). In one example, the subject information includes coordinates indicating the position of each part in the face of the user image 51, the color of each part in the face of the user image 51, and biological information indicating the state of the user (sometimes referred to as vital data). The measurement is performed based on the information (video) acquired by the imaging unit 16, and thus the image processing unit 111 sequentially acquires the biological information without coming in contact with the user.

The following describes coordinate information that is the basis of the measurement processing. The coordinate information includes: information that defines coordinate systems, such as an imaging coordinate system for an image captured by the imaging unit 16 and a display unit coordinate system for the display surface of the display unit 18; and information that indicates the correspondence to be used for conversion of the coordinates in a coordinate system to the coordinates in another coordinate system. Each functional block performs display processing by converting the coordinates in each coordinate system based on the correspondence of the coordinates between the coordinate systems. In one example, the correspondence between these coordinate systems may be set by calibration to correct the correspondence. The calibration includes the adjustment of the direction of the image pickup lens in the imaging unit 16 and the adjustment of the zoom factor during manufacturing the electronic device 1. For example, the zoom factor is adjusted using either or both of an optical zoom by adjusting the lens position of the imaging unit 16 and a digital zoom by image processing.

The video processing unit 111 acquires first pulse-wave amplitude information indicating the average amplitude of the pulse wave of the body within a predetermined time based on the video information of the body in a first video obtained by capturing an image of at least a part of the body, and acquires second pulse-wave amplitude information indicating the average amplitude of the pulse wave of the body within a predetermined time based on the video information of the body in a second video obtained by capturing an image of at least a part of the body after the capturing of the first video.

The above describes that the video processing unit 111 acquires the first pulse-wave amplitude information and second pulse-wave amplitude information indicating the average amplitude of the body pulse wave within a predetermined time, and the present invention is not limited to this. For example, the video processing unit 111 may acquire the first pulse-wave amplitude information and the second pulse-wave amplitude information that is the amplitude information calculated by various methods, such as the most frequent value, the median value, and the average value of the amplitude in a predetermined period.

In one example, when a user performs a massage on the user's own face, the video processing unit 111 obtains the first pulse-wave amplitude information indicating the average amplitude of a pulse wave in the face in a predetermined period from an image of the face that is taken before the massage, and then obtains the second pulse-wave amplitude information indicating the average amplitude of a pulse wave in the face in a predetermined period from an image of the face that is taken after the massage. Note that the “pulse wave” is a single pulse waveform that is detected as the user's biological information, and the biological information can be obtained using the technology described in the following reference, for example.

### REFERENCE

Tohoku University, Cyber Science Center, Advanced Information Technology Research Department, Tohoku University Innovation Research Organization, “Successfully Developed Monitoring Device for Blood Circulation Status” “Magic Mirror””, [online], Sep. 27, 2016, [Search on Dec. 15, 2017], Internet <URL: http://www.tohoku.ac.jp/japanese/newimg/pressimg/tohokuuniv-press20160927_01web.pdf>

The video processing unit 111 includes an imaging condition adjuster 111a.

The imaging condition adjuster 111a acquires the pulse-wave information of the body from the image obtained by capturing an image of at least a part of the body using the measurement camera 25 prior to the imaging of the first video, and adjusts the imaging conditions based on the obtained pulse-wave information. This imaging condition includes at least one of the exposure condition of the measurement camera 25, the amount of illumination used for the measurement camera 25, and the color of the illumination light. The details of the method of adjusting the imaging conditions by the imaging condition adjuster 111a will be described later.

The display processing unit 112 controls to display a motion image as a display video. This allows the user to visually recognize the dynamic blood flow fluctuations and easily understand the differences before and after a specific action. The motion image of the present embodiment is a hue motion image that expresses the measurement result in colors. The hue motion image shows a specific part (first part) that is divided into square-shaped small regions, and blood flow fluctuations in each small region are expressed by changes in hue. The specific part is a part of the face, for example.

The display processing unit 112 also performs synthesis processing that synthesizes a guide image and an avatar image. In one example, the display processing unit 112 controls to make the display unit 18 display a mirror image of the user or an image corresponding to the user (e.g., an avatar). In one example, the display processing unit 112 executes switching processing of switching between a first display mode of displaying the user image as the main and displaying the synthesized image of the avatar image and the guide image as the sub, and a second display mode of displaying the user image as the sub and the synthesized image of the avatar image and the guide image as the main. As illustrated in FIG. 3, the synthesized image of the avatar image and the guide image is displayed in a large size in the center of the screen as the main, and the user image is displayed as a sub in the lower part of the screen in a smaller size than the main image. Conversely, the user image may be displayed in a large size in the center of the screen as the main, and the synthesized image of the avatar image and the guide image may be displayed as a sub in the lower part of the screen in a smaller size than the main image.

The result processing unit 113 compares the first pulse-wave amplitude information with the second pulse-wave amplitude information, and outputs a comparison result indicating the degree of change in blood flow.

In one example, when the user performs a massage on the user's own face, the result processing unit 113 compares the first pulse-wave amplitude information obtained from the image of the face that is captured before the massage and the second pulse-wave amplitude information obtained from the image of the face that is captured after the massage, and outputs a comparison result indicating the degree of change in blood flow in the user's face.

When outputting this comparison result, the result processing unit 113 outputs the comparison result to at least the memory unit 19. As a result, the memory unit 19 stores the history of the comparison results between the first pulse-wave amplitude information and the second pulse-wave amplitude information.

The evaluation processing unit 114 acquires the tendency of the change in blood flow based on the comparison result newly output from the result processing unit 113 and the past comparison result stored in the memory unit 19.

In one example, when the user performs a massage on the user's own face, the evaluation processing unit 114 obtains the tendency of the change in blood flow before and after the massage based on the newly output comparison result and the past comparison result, and evaluates whether or not the massage is continuously performed based on this tendency of the change in blood flow. Specifically, assume the case where the user has already performed the massage using the electronic device 1 at least once, and the memory unit 19 stores the comparison result between the first pulse-wave amplitude information and the second pulse-wave amplitude information before and after the massage. After that, when the user performs the massage again, the evaluation processing unit 114 compares a change in the pulse-wave amplitude between the first pulse-wave amplitude information and the second pulse-wave amplitude information this time with the comparison result stored in the memory unit 19. If the comparison shows the change is the same or less than the last time, the evaluation processing unit 114 determines that the user performs the massage continuously.

The evaluation processing unit 114 may acquire the tendency of the change in blood flow based on the comparison result newly output from the result processing unit 113 and the last and single comparison result stored in the memory unit 19. Alternatively, the evaluation processing unit 114 acquires the tendency of the change in blood flow based on the comparison result newly output from the result processing unit 113 and a plurality of comparison results stored in the memory unit 19. For a plurality of comparison results stored in the memory unit 19, the evaluation processing unit 114 may calculate the maximum and/or minimum and/or average values of the differences between the pulse-wave amplitude of the first pulse-wave amplitude information and the pulse-wave amplitude of the second pulse-wave amplitude information and of the change rates, and may use these calculation results as the plurality of comparison results.

The information processing unit 115 controls settings related to measurement processing and display processing. The information processing unit 115 acquires a measurement result indicating a change in blood flow based on the data analysis by the video processing unit 111, the result processing unit 113, and the evaluation processing unit 114.

In one example, the information processing unit 115 acquires application software for display processing from the application distribution server in the server group 3, and activates this application software. When the user refers to the menu displayed on the display unit 18 and selects any guidance content via the input unit 17, the information processing unit 115 then accepts the selection. In one example, the information processing unit 115 accepts the selection of “esthetic treatment for small-face.” This starts the display processing for guidance of the esthetic treatment for small-face. In one example, “esthetic treatment for small-face” means a lymphatic massage by the user on the user's own face to reduce swelling of the face by massaging to flow the lymph.

In one example, the communication processing unit 116 communicates with the authentication server in the server group 3. This authenticates the user for the display processing. Then, the communication processing unit 116 communicates with the measurement data storage server in the server group 3, for example, to update the profile information of the user in the display processing.

[Video Data Analysis]

First, the acquisition of video data to be analyzed by the video processing unit 111 will be described.

The video processing unit 111 performs processing relating to pattern matching of the contour and parts of the face and face tracking to identify skin colors to recognize the contour of the face, the position of the eyes, and the area of the skin, and detect regions of predetermined parts, such as forehead, cheeks, chin, and neck. In one example, the video processing unit 111 detects the contour of the face and the position of the eyes from the user image 51 in the video, and automatically recognizes a plurality of regions of the forehead, eyelids, cheeks, around the nose, around the lips, chin, neck, and décolleté, based on the relative position from the detected face and eyes. Then the video processing unit 111 detects the states of each of these detected regions, such as the coordinates, the color of the user's skin, and the angle of the face (i.e., the orientation of the user's face) in the user image 51.

To extract a pulse wave from the image, the video processing unit 111 acquires biological information on blood flow such as pulses and pulse waves by utilizing the property that hemoglobin in blood absorbs green light well. The wavelength of green signals is typically 495-570 nm, and hemoglobin has a high absorption coefficient around 500 to 600 nm. When the blood flow increases, the amount of blood on the skin surface increases and the amount of hemoglobin per unit time increases. As a result, more green signals are absorbed by the hemoglobin than before the blood flow increases. This means that the brightness of the green signal detected decreases as the blood flow increases. When the imaging device of the imaging unit 16 converts light into luminance, an RGB filter may be placed in front of the imaging device, and the luminance value of each RGB pixel is calculated. In this case, the light that has passed through the green filter is the luminance value. The sensitivity of the imaging device may not be flat with respect to the wavelength. Even so, the wavelength band can be narrowed down to some extent by the above-mentioned filter, so that the green signal can be detected with high accuracy.

The video processing unit 111 acquires pulse-wave information based on the luminance information included in the video information of the body in the video. Specifically, the video processing unit 111 acquires the brightness of the green signal every unit time, and acquires the pulse-wave information from the change over time (temporal change) in the luminance of the green signal. In one example, the unit time may be the frame rate of a motion image, so that the luminance of the green signal can be acquired for each of the time-continuous images making up the video.

To make it easier to intuitively understand the increase in blood flow, the present embodiment performs conversion processing so that the luminance value increases with the blood flow. Specifically, when detecting the luminance of a green signal using an image sensor that outputs 8-bit for each of RGB colors, the “converted luminance” is obtained by subtracting the detected luminance value of green signal from the maximum luminance value of 255, and the obtained value is used for comparison processing. Hereinafter, the value simply described as the converted luminance is the information on the luminance underwent such a conversion.

To compare the state of blood flow before and after the event by the user, the electronic device 1 captures images of the user before the event to acquire a first video, and also captures images of the user after the event to acquire a second video.

Examples of the event include various beauty-related treatments such as massage to promote blood flow and application of skin cream that promotes blood circulation, and various types of activities such as exercise, including sports and relaxation, from which a change in blood flow can be expected.

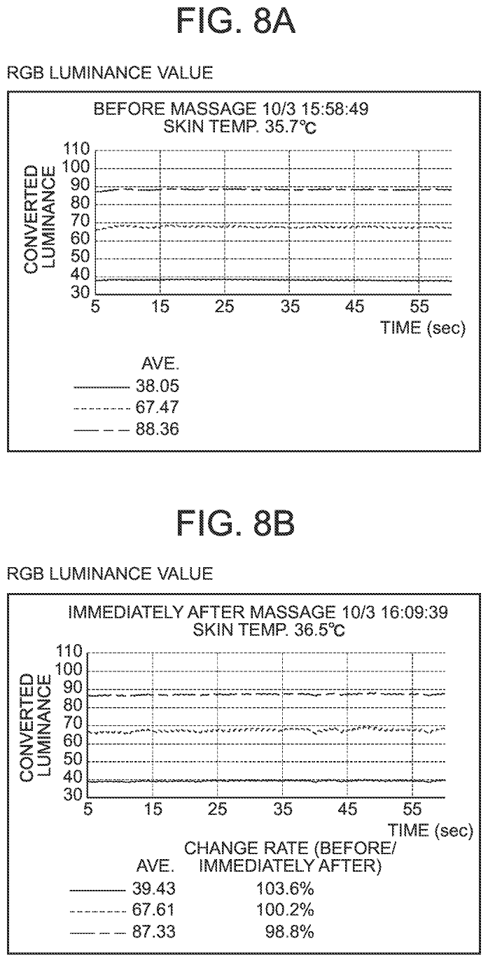

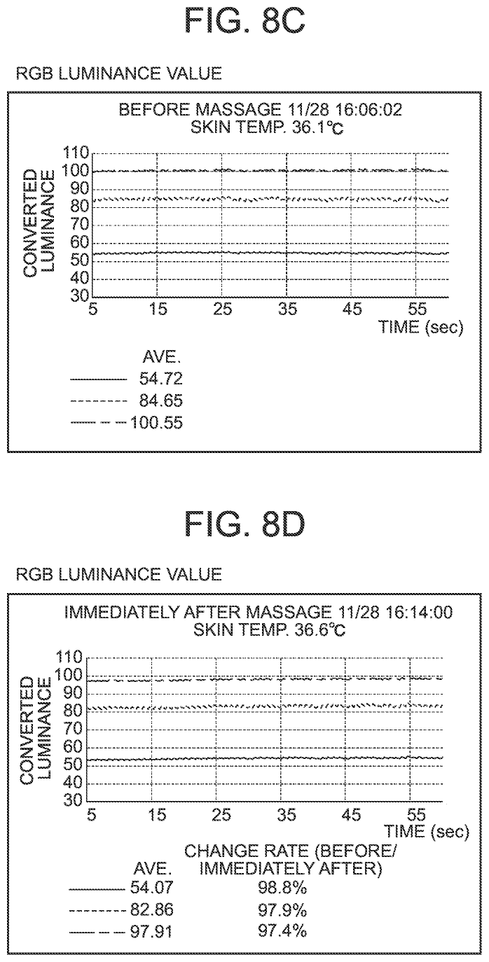

FIGS. 8A to 8D illustrate the temporal change of the RGB luminance value in the first video before and after the massage as an event, and the temporal change of the RGB luminance value in the second video before and after the massage.

Specifically, FIG. 8A illustrates an example of the temporal change of the RGB luminance values before the massage starting on October 3. FIG. 8B illustrates the temporal change of the RGB luminance values after the massage on the same date, October 3. FIG. 8C illustrates the temporal change of the RGB luminance values before the massage on November 28 after about two months from the time of FIG. 8A and FIG. 8B. FIG. 8D illustrates the temporal change of the RGB luminance values after the massage on the same date, November 28.

The light that hits the skin is absorbed by hemoglobin in the blood of the capillaries at the epidermis, so that the reflected light is weakened. The present embodiment uses a converted luminance value, so that a larger luminance value means lower luminance, and accordingly more blood flow. The RGB luminance values in the graph reflect the luminance of the RGB signals at multiple locations of the measured portion, and they are calculated by various methods such as the most frequent value, the median value, and the average value. This embodiment indicates the temporal change of the average of the luminance for the RGB signals of all the pixels of the measured portion.

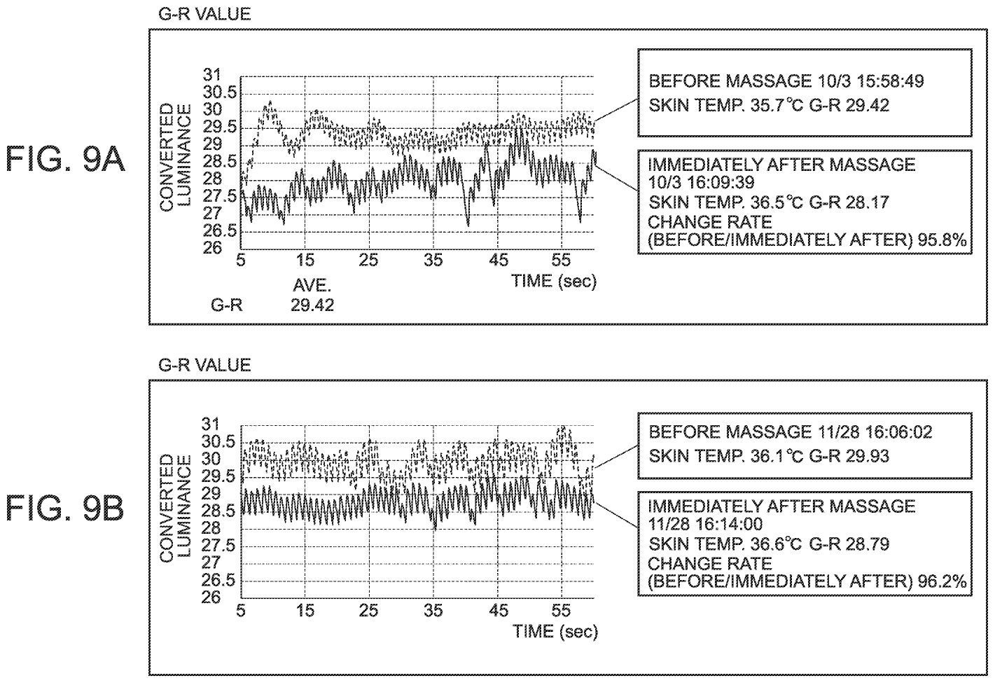

To suppress the effects from disturbance, FIGS. 9A and 9B illustrate the values obtained by subtracting the temporal change of the luminance value of the R signal in FIG. 8 from the temporal change of the luminance value of the G signal in FIG. 8.

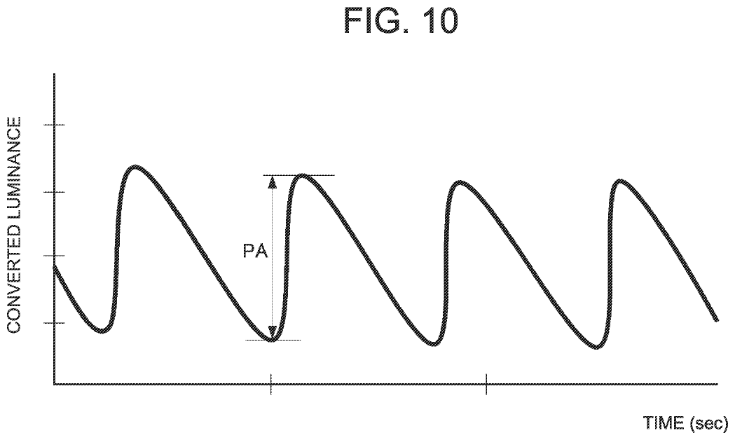

Note here that the pulse wave has an amplitude. FIG. 10 is a graph schematically illustrating the pulse-wave amplitude PA or pulse amplitude measured by the electronic device 1 of one embodiment of the present invention. As illustrated in FIG. 10, the pulse wave analyzed from the video shows a periodic waveform within a range of a constant amplitude PA. The amplitude PA of this pulse wave means the difference between the adjacent maximum and minimum values of the pulse wave signal. The range for acquiring the amplitude PA is preferably a region without abnormal values and having stable amplitude. In one example, if an abnormal value exceeding a preset threshold is detected, pulse wave information is acquired so as not to include the abnormal value. Alternatively, it may be displayed that acquisition of appropriate image failed during imaging, and then re-imaging may be performed to acquire appropriate pulse wave information. Alternatively, the pulse wave after a predetermined time has elapsed from the start of imaging may be used to calculate the amplitude. Alternatively, the amplitude may be calculated by removing an abnormal value from the pulse wave acquired within a predetermined time. In this way, various methods can be used to calculate the amplitude.

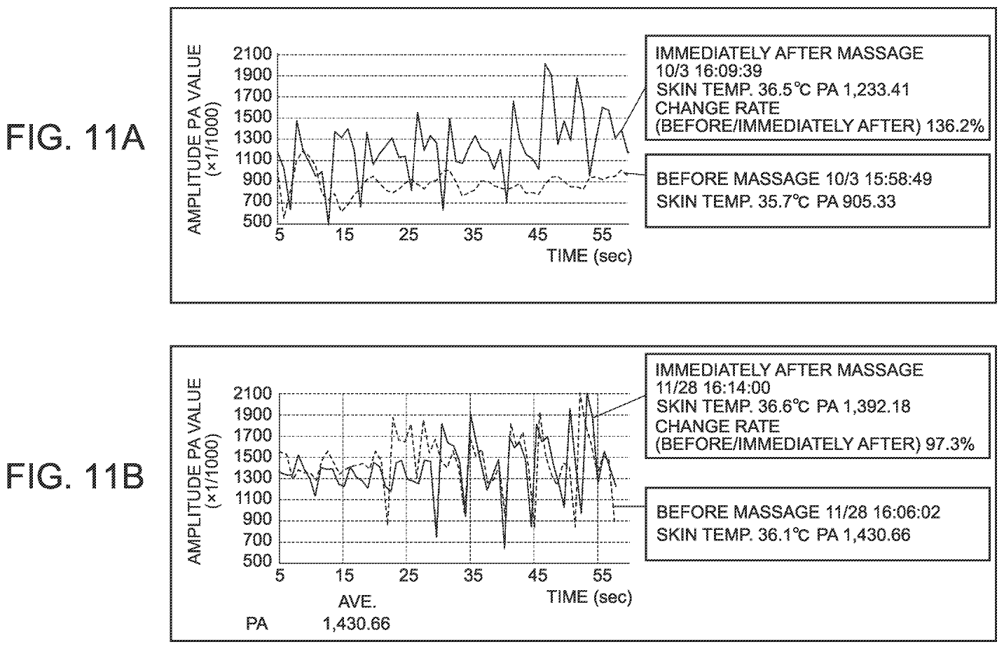

FIGS. 11A and 11B illustrate the temporal change of the amplitude PA in the first video before and after the massage as an event, and the temporal change of the amplitude PA in the second video before and after the massage. Specifically, FIG. 11A corresponds to FIG. 9A, and FIG. 11B corresponds to FIG. 9B.

As can be understood from FIGS. 8, 9, and 11, the data of October 3, when the massage was started, shows that the G luminance values and G-R values did not change in luminance values before and after the massage. Specifically, for the G luminance value, the rate of change of the value after the massage is 100.2% relative to the value before the massage. For the G-R value, the rate of change of the value after the massage is 95.8% relative to the value before the massage. In contrast, comparison of the amplitude PA values shows that the amplitude increased significantly. Specifically, the rate of change of the value after the massage is 136.2% relative to the value before the massage.

The data of November 28 about two months later from the start of the massage shows that the G luminance values and G-R values did not change before and after the massage, and comparison of the amplitude PA values also did not change. Specifically, for the G luminance value, the rate of change of the value after the massage is 97.9% relative to the value before the massage. For the G-R value, the rate of change of the value after the massage is 96.2% relative to the value before the massage. For the amplitude PA value, the rate of change of the value after the massage is 97.3% relative to the value before the massage.

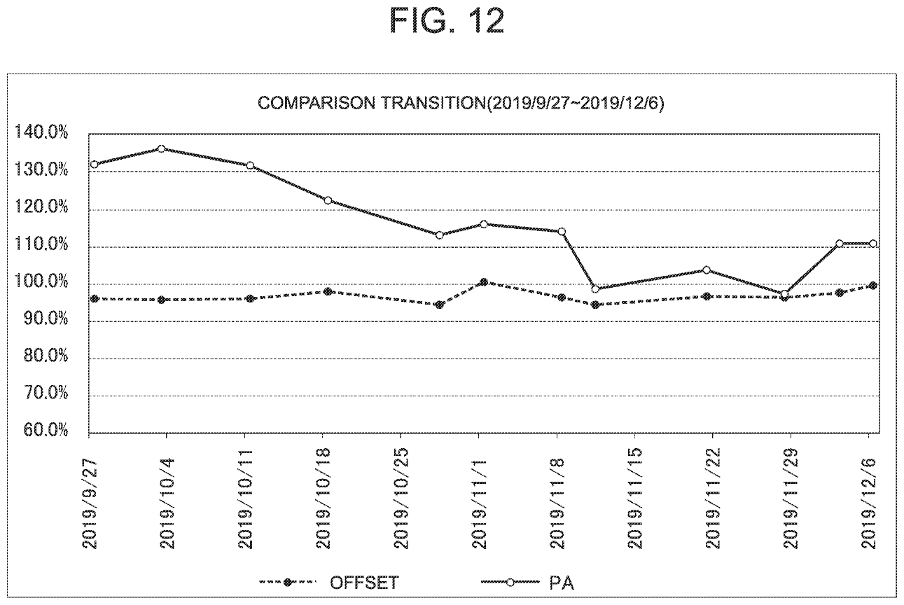

FIG. 12 is a graph showing the transition of the rate of change of the measured values measured by the measurement camera 25 for the amplitude PA value and the blood-flow baseline offset value during the time period when massage is performed for a long term.

As mentioned above, at the beginning of start of the massage, the amplitude PA value after the massage increases significantly compared to the amplitude PA value before the massage. Then as days passes from the start of the massage, the change rate of the amplitude PA value after the massage to the amplitude PA value before the massage decreases. As illustrating in FIG. 12, the change in the amplitude PA value shows the effect of a treatment such as a massage for a long term, indicating that the expansion of the skin capillaries can be maintained due to a correct massage.

More specifically, when a massage is performed for a long term as the treatment, the measured change rate of the amplitude PA value of the image pulse wave before and after the treatment gradually decreases. This is because capillaries are dilated by the external pressure of the massage, and the continuous massage further keeps the dilatation, so that the change rate decreases. The electronic device 1 determines whether or not the massage is performed correctly based on this change rate (decreasing value) to determine whether or not the optimum massage continues. The electronic device 1 then notifies the user whether or not an appropriate massage is performed, by giving appropriate notifications and guidance instructions according to those values. If there is no decrease in the amplitude PA value, the electronic device 1 notifies the user that a proper massage is not performed and gives guidance on the proper massage.

In this way, the electronic device 1 determines the effect of the treatment based on the transition illustrated in FIG. 12 and appropriately notifies the user of the effect. This allows the user to keep the motivation required for self-care, and at the same time, perform the treatment while feeling the effect. As a result, the user can perform the treatment continuously for a long period of time to promote the smooth turnover of the skin.

To increase the accuracy of determination, the electronic device 1 may record both of the time and the frequency since the start of the treatment, and may change the change rate as the standard as in 120% or more for the first month, 110% or more for the second month, and 90% or more for the third month or later, for example.

The electronic device 1 may be configured to change the degree of effectiveness for each age group. In one example, the determination may be made so that for the user in twenties, the standard range of change rate is 90% to 140%, and for the user in 60 years old or older, the standard range is 90% to 110%.

The determination may differ according to genders, together with the age groups mentioned above. In one example, the change rate for men may decrease by 10%, and the change rate for women may increase by 10%.

The determination standard may be set according to the skin type of the user. This can be implemented by combining with a function of inspecting the skin type of the user in advance. In one example, for the user having a young skin type (skin age is young), the determination standard may increase by 10% relative to the standard (e.g., 90%-130%), and for the user having an aging skin type (skin age is old), it may decrease by 10% relative to the standard range.

Further, data may be stored in the cloud, and machine learning of the data may be conducted by cluster processing that classifies the data into groups of similar ones, based on the data that are saved with tags of actual ages and genders. This creates a classifier to create the determination standard.

[Adjustment of Imaging Conditions]

Next, the following describes the adjustment of imaging conditions by the imaging condition adjuster 111a.

As described above, the video processing unit 111 acquires video data. At this time, when the skin from which the video data is acquired is relatively fair, it may be difficult to obtain a pulse wave signal due to the difference in skin structure from the horny layer, the epidermis and the dermis to the capillaries of the skin.

Then the imaging condition adjuster 111a acquires pulse wave information from the video data acquired by the video processing unit 111 and adjusts the imaging conditions based on this pulse wave information.

Specifically, if at least one of the pulse wave amplitude PA value indicating the blood flow rate, the offset value indicating the average blood flow rate (G luminance signal value or the value obtained by G luminance signal-R luminance signal), and the signal to noise (SN) ratio indicating the ratio between pulse wave frequency and other signals, which are derived from the acquired pulse wave information, exceeds their predetermined thresholds, the imaging condition adjuster 111a does not change the imaging conditions, and acquires the first pulse-wave amplitude information or the second pulse-wave amplitude information using the acquired pulse wave information.

In contrast, if all of the pulse wave amplitude PA value indicating the blood flow rate, the offset value indicating the average blood flow rate (G luminance signal value or the value obtained by G luminance signal-R luminance signal), and the signal to noise (SN) ratio indicating the ratio between pulse wave frequency and other signals, which are derived from the acquired pulse wave information, fall below their predetermined thresholds, the imaging condition adjuster 111a changes the imaging conditions.

The “imaging conditions” relate to the exposure of the measurement camera 25, the light intensity of the LED of the measurement camera 25, and the color of the light emitted from this LED.

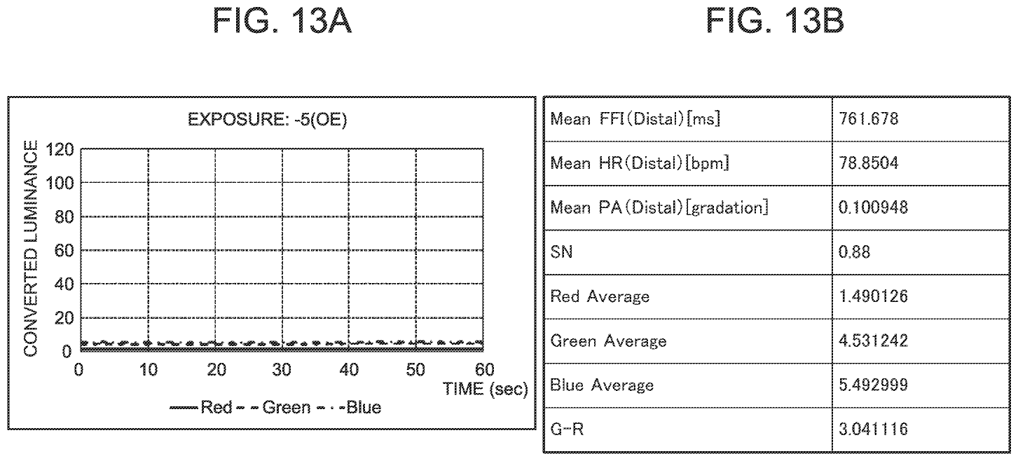

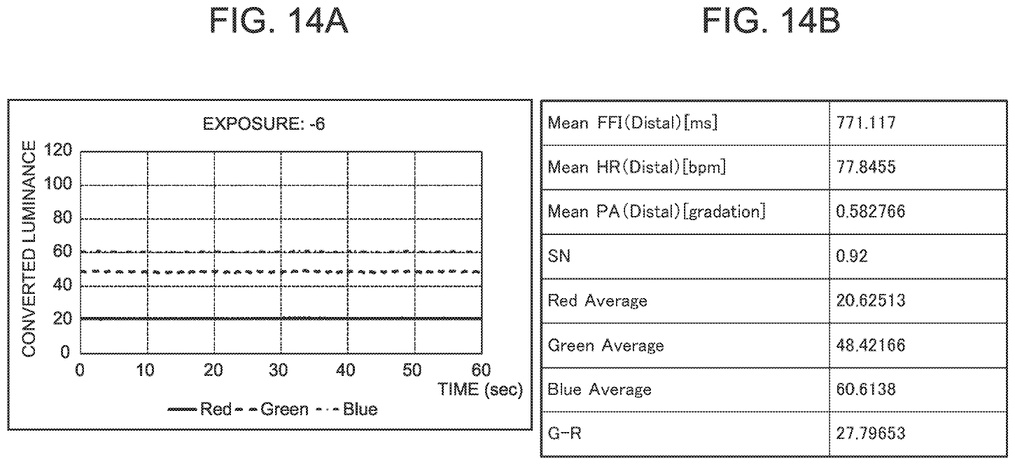

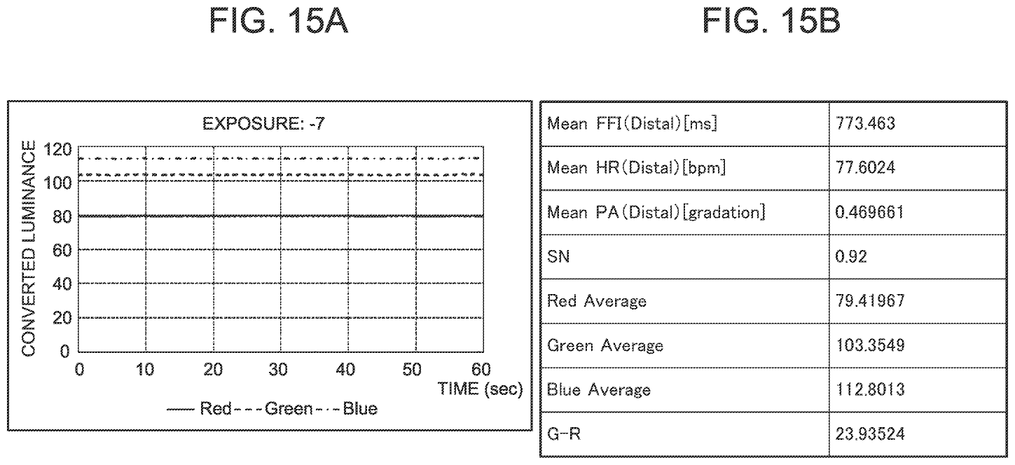

FIGS. 13 to 15 illustrate examples of graphs indicating the RGB luminance value, the G luminance value, and the pulse-wave amplitude and tables of measurements for different exposures of the measurement camera 25. Specifically, FIG. 13 is an example of the RGB luminance values and the table of the measurements when the exposure is −5 as the initial setting. FIG. 14 is an example of the RGB luminance values and the table of the measurements when the exposure is −6. FIG. 15 is an example of the RGB luminance values and the table of the measurements when the exposure is −7.

For example, FIG. 13A is a graph of the temporal change of the RGB luminance value. FIG. 13B indicates various measurements when the exposure is −5, and the calculated values calculated from the graph of FIG. 13A. “Mean FFI” is the value of pulse wave fluctuation. “Mean HR” is the pulse rate. “Mean PA” is the amplitude PA value. “SN” is the signal-to-noise ratio. “Red Average” is the average of R luminance values. “Green Average” is the average of G luminance values. “Blue Average” is the average of B luminance values. “G-R” is the value of G-R.

Comparison of the values in FIG. 13B, FIG. 14B, and FIG. 15B indicates that, although the SN ratios in FIG. 14B and FIG. 15B have the same values, the exposure of −6 is the optimum value because the amplitude PA value, SN ratio, and G-R value are almost the maximum in FIG. 14B having the exposure being −6.

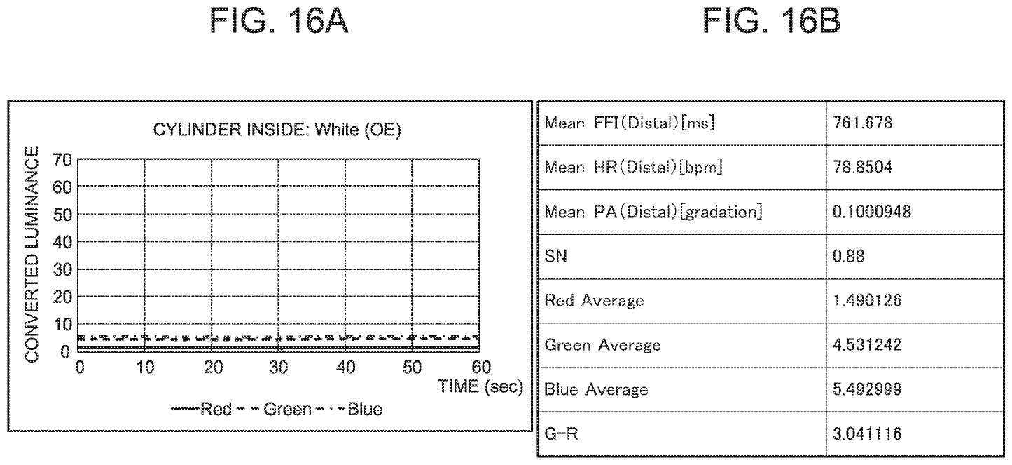

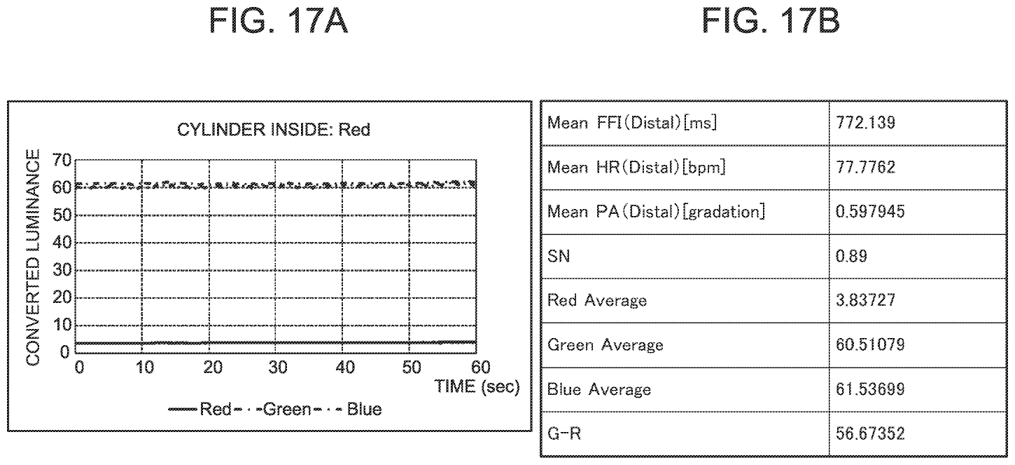

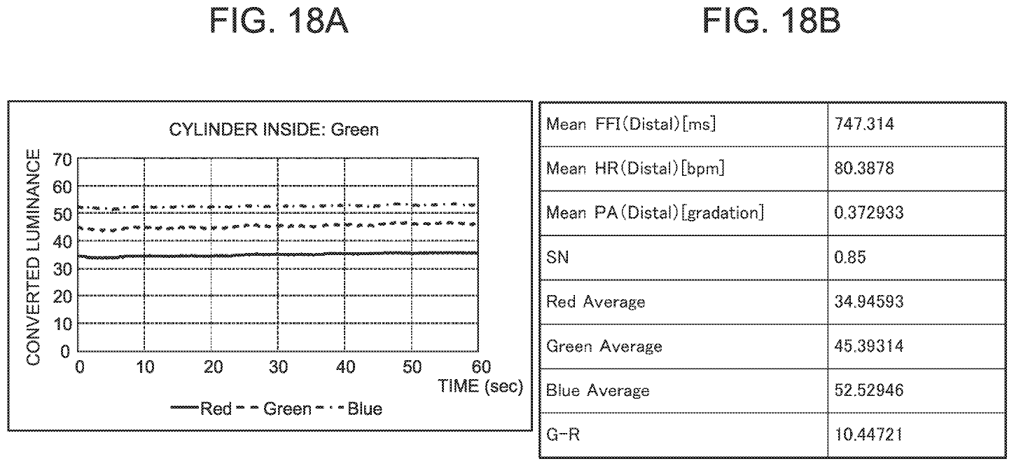

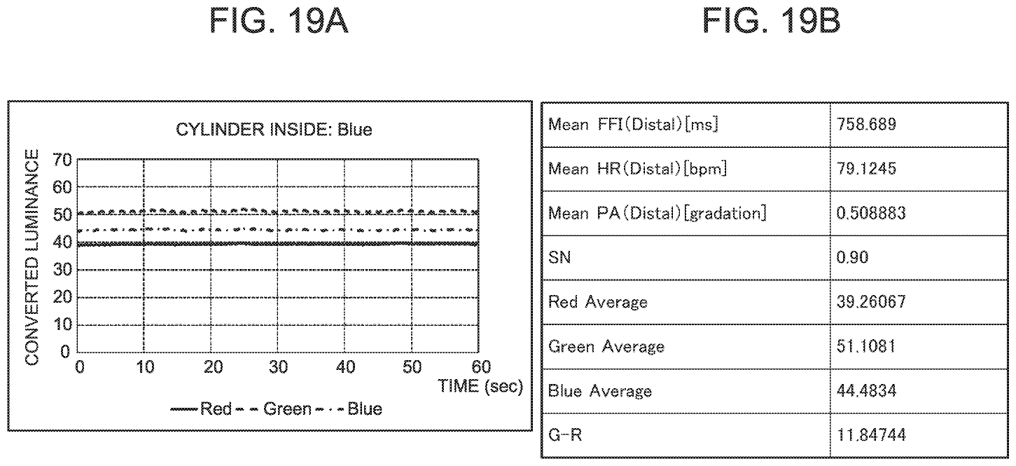

FIGS. 16 to 19 illustrate examples of graphs indicating the RGB luminance value, the G luminance value, and the pulse-wave amplitude and tables of measurements when the color of the light emitted from the LED that is the illumination unit 257 is varied by changing the color of the inner wall of the cover 252 of the measurement camera 25. Specifically, FIGS. 16A and 16B are an example of the graph indicating the RGB luminance values, the G luminance value and the pulse-wave amplitude and the table of the measurements when the color of the inner wall is white as the initial setting. FIGS. 17A and 17B are an example of the graph indicating the RGB luminance values, the G luminance value and the pulse-wave amplitude and the table of the measurements when the color of the inner wall is red. FIGS. 18A and 18B are an example of the graph indicating the RGB luminance values, the G luminance value and the pulse-wave amplitude and the table of the measurements when the color of the inner wall is green. FIGS. 19A and 19B are an example of the graph indicating the RGB luminance values, the G luminance value and the pulse-wave amplitude and the table of the measurements when the color of the inner wall is blue.

Similar to FIG. 13A, FIG. 16A is a graph of the temporal change of the RGB luminance value. FIG. 16B indicates various measurements when the inner wall is white, and the calculated values calculated from the graph of FIG. 16A.

Comparison between the values of FIGS. 16B, 17B, 18B, and 19B indicates that the amplitude PA value is the maximum of 0.597945 when the color of the inner wall is red illustrated in FIG. 17B. The comparison also indicates that the SN ratio is the maximum of 0.90 when the color of the inner wall is blue illustrated in FIG. 19B. The comparison also indicates that the G-R value is the maximum of 56.67352 when the color of the inner wall is red illustrated in FIG. 17B. In one example, assume the case where the amplitude PA value or the G-R value is preferentially used as the condition for comparison with the threshold. Then the determination will be that the optimum color of the inner wall is red, i.e., red is the optimum color for the light to be emitted from the LED.

The above describes the example where, if at least one of the pulse wave amplitude PA value indicating the blood flow rate, the offset value indicating the average blood flow rate, and the SN ratio indicating the ratio between pulse wave frequency and other signals, which are derived from the acquired pulse wave information, exceeds their predetermined thresholds, the imaging condition adjuster 111a does not change the imaging conditions, and acquires the first pulse-wave amplitude information or the second pulse-wave amplitude information using the acquired pulse-wave information. The present invention is not limited to this example. In another example, if a plurality of the pulse wave amplitude PA value indicating the blood flow rate, the offset value indicating the average blood flow rate, and the SN ratio indicating the ratio between pulse wave frequency and other signals, which are derived from the acquired pulse wave information, exceeds their predetermined thresholds at the same time, the imaging condition adjuster 111a does not change the imaging conditions, and may acquire the first pulse-wave amplitude information or the second pulse-wave amplitude information using the acquired pulse-wave information.

In another example, the imaging condition adjuster 111a may set any priority for the pulse wave amplitude PA value indicating the blood flow rate, the offset value indicating the average blood flow rate, and the SN ratio indicating the ratio between pulse wave frequency and other signals, which are derived from the acquired pulse wave information and are the targets of the comparison. In one example, when the amplitude PA value exceeds a first threshold, the imaging condition adjuster 111a may compare at least one of the offset value and the SN ratio with a second threshold. In another example, when the offset value exceeds a first threshold, the imaging condition adjuster 111a may compare at least one of the amplitude PA value and the SN ratio with a second threshold.

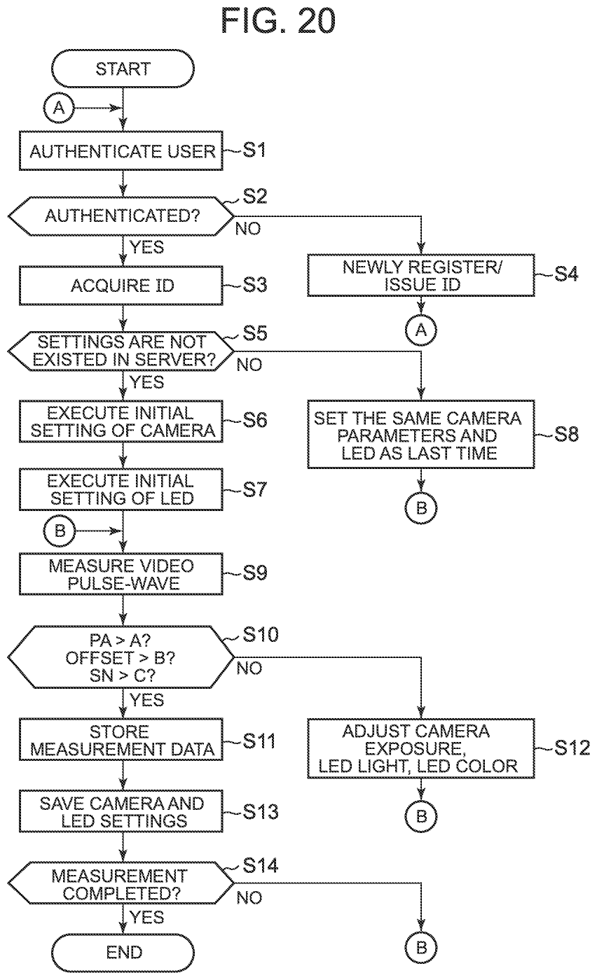

Referring next to FIG. 20, the following describes the flow of processing to adjust the imaging conditions. FIG. 20 is a flowchart describing a flow of the processing executed by the electronic device 1 of FIG. 1 having the functional configuration of FIG. 7.

In step S1, the electronic device 1 communicates with the authentication server 3 to identify the user by performing personal authentication such as password authentication, face authentication, or fingerprint authentication.

In step S2, if the user is authenticated (S2: YES), the procedure shifts to step S3. If the user is not authenticated (S2: NO), the procedure shifts to step S4.

In step S3, the electronic device 1 acquires an ID from the authentication server 3. This ID is used for managing measurement data on the cloud.

In step S4, the electronic device 1 performs personal registration and issues an ID. The procedure then shifts to step S1.

In step S5, if the previous parameter setting information on the measurement camera 25 and the LED associated with the acquired ID does not exist in the server or the like (S5: YES), the procedure shifts to step S6. If the previous parameter setting information on the measurement camera 25 and the LED associated with the acquired ID exists in the server or the like (S5: NO), the procedure shifts to step S8.

In step S6, the electronic device 1 executes initial setting of the measurement camera 25 using the default parameters with the measurement application installed in the electronic device 1. Examples of the “default parameters” include parameters such as camera exposure, focal length, white balance, and autofocus ON/OFF.

In step S7, the electronic device 1 executes initial setting of the LED using the default parameters with the measurement application installed in the electronic device 1. Examples of the “default parameters” include parameters such as light intensity of the LED and the color of the light emitted from the LED (e.g., white light).

In step S8, the electronic device 1 configures the measurement camera 25 and the LED based on the parameter setting information including the parameters set last time. The procedure then shifts to step S9.

In step S9, the electronic device 1 measures video pulse-wave data with the measurement application installed in the electronic device 1. The video pulse-wave data contains the offset value indicating the average blood flow rate (G luminance value or G luminance value-R luminance value), the pulse wave amplitude PA value indicating the blood flow rate, and the SN ratio indicating the ratio between pulse wave frequency and other signals.

In step S10, the electronic device 1 determines the acquired data by the following expressions:

Amplitude PA value>A;

Offset value>B;

SN value>C.

A, B, and C, which are the criteria for determination, are set so that the amplitude PA value and offset value are the maximum and the SN value is 70% or more. The SN value of 70% or more means that the amplitude PA value and the offset value can be determined at a reliable level.

In step S10, if any one of the above formulas is satisfied (S10: YES), the procedure shifts to step S11. If none of the above formulas are satisfied (S10: NO), the procedure shifts to step S12.

In step S11, the electronic device 1 saves the measurement data for each ID to the server, for example, and analyzes the data. The user of the electronic device 1 sees the transition of the data so as to utilize this to improve beauty conditions, such as for the skin, based on the blood flow, and improve health conditions using the data for health care information such as blood pressure fluctuation and autonomic nerve index based on the value of the fluctuation of the pulse wave and the pulse rate.

In step S12, the electronic device 1 adjusts various types of parameters. In one example, when adjusting the camera parameters of the measurement camera 25, the electronic device 1 adjusts the exposure. For example, in the case of exposure −5 illustrated in FIG. 15, the amount of light is large, so the electronic device 1 adjusts the exposure by −1 and sets to −6 shown in FIG. 16. For example, in the case of exposure −7 illustrated in FIG. 17, the amount of light is large, so the electronic device 1 adjusts the exposure by +1 and sets to −6 shown in FIG. 16. In this case, similar results can be obtained by increasing or decreasing the amount of LED light, in addition to the camera parameters.

In one example, when adjusting the light emitted from the LED, the electronic device 1 adjusts the color of light. For example, in the cases illustrated in FIGS. 16 to 19, red has the maximum amplitude PA value, offset value, and SN value, and the electronic device 1 therefore adjusts the LED to emit red light.

The procedure then shifts to step S9.

In step S13, the electronic device 1 saves various types of parameter information on the settings of the measurement camera 25 and the LED to a server, for example, for each ID so that the measurement can be performed with the same settings in the following measurements.

In step S14, when the measurement is completed (S14: YES), the entire procedure ends. If the measurement is not completed (S14: NO), the procedure shifts to step S9.

The following describes the advantageous effects from the electronic device 1 of the present embodiment.

The electronic device 1 of this embodiment includes the imaging condition adjuster 111a and the video processing unit 111. The imaging condition adjuster 111a acquires the pulse-wave information for adjustment indicating pulse waves of the body from the video obtained by capturing an image of at least a part of the body using the imaging device, and adjusts the imaging conditions based on the obtained pulse-wave information for adjustment. The video processing unit 111 makes the imaging device capture a first video of at least a part of the body based on the imaging conditions adjusted by the imaging condition adjuster 111a, and acquires first pulse-wave information on the body based on the video information on the body in the first video.

As a result, even when the user's skin is relatively fair, the electronic device accurately measures the effect of continuous beauty treatment based on the biological information acquired from the video. Specifically, when the skin is relatively fair and it is difficult to obtain biological information, the electronic device adjusts the amount and color of LED lighting, for example, for measurement in an appropriate range, and also prevents the SN ratio from dropping due to noise caused by a slight movement of the skin or noise caused by a movement of the camera body during installation for skin measurement. The electronic device therefore suppresses a measurement error due to a difference in skin color.

The imaging conditions include at least one of the exposure condition of the imaging device, the amount of illumination used for the imaging device, and the color of the illumination light.

The electronic device adjusts the exposure condition of the imaging device, the amount of illumination used for the imaging device, and the color of the illumination light in this way, so that even when the user's skin is relatively fair, the electronic device accurately measures the effect of continuously performed beauty treatment based on the biological information acquired from the video.

The imaging condition adjuster 111a sets the imaging conditions based on the comparison result of at least one of the pulse wave amplitude value indicating the blood flow rate, the offset value indicating the average blood flow rate, and the SN ratio indicating the ratio between pulse wave frequency and other signals with their thresholds.

In this way, even when the user's skin is relatively fair, the electronic device accurately measures the effect of continuous beauty treatment based on the biological information acquired from the video and based on at least one of the pulse wave amplitude value indicating the blood flow rate, the offset value indicating the average blood flow rate, and the SN ratio indicating the ratio between pulse wave frequency and other signals.

When the amplitude value exceeds a first threshold, the imaging condition adjuster 111a compares at least one of the offset value and the SN ratio with a second threshold.

This makes it possible to preferentially use the amplitude value as the comparison condition.

When the offset value exceeds a first threshold, the imaging condition adjuster 111a compares at least one of the amplitude value and the SN ratio with a second threshold.

This makes it possible to preferentially use the offset value as the comparison condition.

When the SN ratio exceeds a first threshold, the imaging condition adjuster 111a compares at least one of the amplitude value and the offset value with a second threshold.

This makes it possible to preferentially use the SN ratio as the comparison condition.

When the amplitude value exceeds a first threshold, the offset value exceeds a second threshold, and the SN ratio exceeds a third threshold, the imaging condition adjuster 111a sets the imaging condition without changing it.

As a result, even when the user's skin is relatively fair, the electronic device more accurately measures the effect of continuous beauty treatment based on the biological information acquired from the video.

The imaging device includes the imaging unit 256 that captures an image of a measurement target, the cylindrical cover 252 that isolates the imaging unit 256 from the outside, and the illumination unit 257 that irradiates the measurement target with light inside the cover 252.

The imaging device captures an image of the measurement target while keeping the distal end of the cover 252 in contact with the measurement target. This keeps the brightness inside the cover constant and so enables the acquisition of a video while suppressing the influence from disturbance.

### Modified Examples

The present invention is not limited to the above embodiment, and may include any modification and improvement as long as such modification and improvement are compatible with an object of the invention. For example, the above-described embodiment may be modified as in the following examples.

The above embodiment describes the configuration in which the comparison processing is performed using the converted luminance values that have underwent the conversion of the detected luminance. The present invention is not limited to this configuration. The converted luminance value is one example indicating the level of the luminance. The above embodiment may omit the conversion processing, and may use the detected luminance value without conversion.

The display unit 18 of the electronic device 1 of the above embodiment may be combined with a mirror portion having a reflective surface. In this case, the mirror portion is implemented with a half mirror having both transmission characteristics and reflection characteristics as optical characteristics. Then, the mirror portion is placed to be superimposed in front of the display unit 18 so that the user visually recognizes the mirror portion. Such an arrangement allows the user to visually recognize not a user image captured by the imaging unit 16, but both their face reflected by the mirror portion and various information displayed on the display unit 18 and transmitted through the mirror portion (e.g., a composite image). That is, the above-described embodiment is configured to display a user image captured by the imaging unit 16 as the user's real image. In this modified example, the user sees their mirror image reflected by the mirror unit as the real image. Such a configuration also leads to the same advantageous effects as those described in the above embodiment.

### Other Modified Examples

The above-described embodiment assumes the case of the electronic device 1 cooperating with the servers in the server group 3. In another example, the electronic device 1 may additionally have the functions of these servers to perform all of the processing by itself.

The above embodiment describes the example of the electronic device 1 that is incorporated into a self-supporting portable mirror. The present invention is not limited to this example. In another example, the present invention is applicable to an electronic device incorporated in a large mirror, such as a full-length mirror, an electronic device incorporated in a stationary bathroom vanity, and an electronic device having a mirror shape installed in bathroom.

The above-stated series of processing may be executed by hardware or by software. In other words, the functional configuration of FIG. 7 is illustrative, and is not limited particularly. That is, the electronic device 1 may have a function of executing the above-stated series of processing as a whole, and the functional blocks to implement the function are not limited particularly to the example of FIG. 7.

One of the functional blocks may be configured with a single hardware unit or with a single software unit, or may be configured with a combination thereof. The functional configuration of the present embodiment is implemented by a processor configured to execute the arithmetic processing. Such a processor that can be used in the present embodiment includes various types of processors as a single unit including a single processor, a multi-processor, and a multicore processor as well as a combination of these various types of processors and a processing circuit, such as an application specific integrated circuit (ASIC) or a field programmable gate array (FPGA).

When a series of processing is executed with software, a program configuring the software may be installed into a computer, for example, via a network or from a recording medium.

The computer may be a computer incorporated into a dedicated hardware. The computer may be a computer capable of executing various types of functions by installing various types of programs in the computer, and may be a general-purpose personal computer, for example.

A recording medium containing such a program may be configured with the removable medium 100 of FIG. 5 that is distributed separately from the main body of the device to provide the program to a user. Alternatively, the recording medium may be provided to a user while being incorporated beforehand into the main body of the device. The removable medium 100 may be configured as a magnetic disk including a floppy disk, an optical disk, an magnetic optical disk or the like. The optical disk may be configured as a CD-ROM (Compact Disk-Read Only Memory), a DVD, a Blu-ray (registered trademark), a Disc (Blu-ray disc) or the like. The magnetic optical disk may be configured as a mini-disk (MD) or the like. The recording medium that is provided to a user while being incorporated beforehand into the body of the device may be configured as the ROM 12 of FIG. 5 containing a program, the hard disk included in the memory unit 19 of FIG. 5, or the like.

In the present specification, the steps describing the programs recorded on the recording medium includes the processing that is performed in a time series manner according to the recorded order. The processing is not necessarily performed in a time series manner, and the steps also include the processing that is performed in a parallel or independent manner. In the present specification, the term of system means an entire device including a plurality of devices and a plurality of means.

That is the description of some embodiments of the present invention. These embodiments are just illustrative, and the technical scope of the present invention is not limited to those examples. The present invention can be in the form of other various embodiments, and may include any modifications such as omission and substitution without departing from the scope of the present invention. The scope and the spirit of the invention described in the present specification as well as the accompanying claims and their equivalents cover these embodiments and modifications thereof.

What is claimed is:

1. An electronic device comprising:
a memory that stores a program; and
at least one processor configured to execute the program stored in the memory,
the at least one processor being configured to
adjust an imaging condition including at least one of an exposure condition of an imaging device, an amount of illumination used for the imaging device and a color of light for the illumination, based on pulse-wave information for adjustment indicating a pulse wave acquired from a video of at least a part of a subject's body captured by the imaging device, and
acquire first pulse-wave information on the subject's body based on video information on the subject's body in a first video that is acquired by capturing the at least a part of the subject's body under the adjusted imaging condition.

2. The electronic device according to claim 1, wherein
the at least one processor sets the imaging condition based on a comparison result of at least one of an amplitude value of the pulse wave indicating a blood flow rate, an offset value indicating an average blood flow rate, and a SN ratio indicating a ratio between pulse wave frequency and other signals with corresponding thresholds.

3. The electronic device according to claim 2, wherein in response to the amplitude value exceeding a first threshold, the at least one processor compares at least one of the offset value and the SN ratio with a second threshold.

4. The electronic device according to claim 2, wherein in response to the offset value exceeding a first threshold, the at least one processor compares at least one of the amplitude value and the SN ratio with a second threshold.

5. The electronic device according to claim 2, wherein in response to the SN ratio exceeding a first threshold, the at least one processor compares at least one of the amplitude value and the offset value with a second threshold.

6. The electronic device according to claim 2, wherein in response to the amplitude value exceeding a first threshold, the offset value exceeding a second threshold, and the SN ratio exceeding a third threshold, the at least one processor sets the imaging condition without changing the imaging condition.

7. The electronic device according to claim 1, wherein the imaging device comprises:
a camera configured to capture an image of a measurement target;
a cylindrical cover that isolates the camera from the outside; and
an illumination unit configured to irradiate a measurement target with light inside the cover.

8. A control method for an electronic device executed by a computer including at least one processor, the control method causing the at least one processor to execute a program stored in a memory to perform operations comprising:
adjusting an imaging condition including at least one of an exposure condition of an imaging device, an amount of illumination used for the imaging device and a color of light for the illumination, based on pulse-wave information for adjustment indicating a pulse wave acquired from a video of at least a part of a subject's body captured by the imaging device, and
acquiring first pulse-wave information on the subject's body based on video information on the subject's body in a first video that is acquired by capturing the at least a part of the subject's body under the adjusted imaging condition.

9. The control method for an electronic device according to claim 8, further comprising setting the imaging condition based on a comparison result of at least one of an amplitude value of the pulse wave indicating a blood flow rate, an offset value indicating an average blood flow rate, and a SN ratio indicating a ratio between pulse wave frequency and other signals with corresponding thresholds.

10. The control method for an electronic device according to claim 9, further comprising in response to the amplitude value exceeding a first threshold, comparing at least one of the offset value and the SN ratio with a second threshold.

11. The control method for an electronic device according to claim 9, further comprising in response to the offset value exceeding a first threshold, comparing at least one of the amplitude value and the SN ratio with a second threshold.

12. The control method for electronic device according to claim 9, further comprising in response to the SN ratio exceeding a first threshold, comparing at least one of the amplitude value and the offset value with a second threshold.

13. The control method for an electronic device according to claim 9, further comprising in response to the amplitude value exceeding a first threshold, the offset value exceeding a second threshold, and the SN ratio exceeding a third threshold, setting the imaging condition without changing the imaging condition.

14. A non-transitory computer-readable storage medium storing a program that is executed by a computer that at least one processor to control an electronic device, the program being executable to cause the computer to perform operations comprising:
adjusting an imaging condition including at least one of an exposure condition of an imaging device, an amount of illumination used for the imaging device and a color of light for the illumination, based on pulse-wave information for adjustment indicating a pulse wave acquired from a video of at least a part of a subject's body captured by the imaging device, and
acquiring first pulse-wave information on the subject's body based on video information on the subject's body in a first video that is acquired by capturing the at least a part of the subject's body under the adjusted imaging condition.
